(19) United States
(12) Patent Application Publication
(10) Pub. No.: US 2006/0148848 A1
(43) Pub. Date: Jul. 6, 2006

(54) Compounds and methods for treating cancer and inflammation

(75) Inventors: Zaihui Zhang, Vancouver (CA); David L Charest, Vancouver (CA); Jun Yan, Coquitlam (CA)

Correspondence Address: SEED INTELLECTUAL PROPERTY LAW GROUP PLLC, 701 FIFTH AVE, SUITE 6300, SEATTLE, WA, 98104-7092, US

(73) Assignee: QLT, Inc.

(21) Appl. No.: 10/520,250

(22) PCT Filed: Jun. 25, 2003
(86) PCT No.: PCT/CA03/00975

§ 371 Date: Oct. 28, 2005

Related U.S. Application Data
(60) Provisional application No. 60/393,700, filed on Jul. 2, 2002.

(51) Int. Cl. A61K 31/47

(52) U.S. Cl. 514/309

(57) ABSTRACT

Methods of using isoquinolone derivatives to treat cancer or inflammation in a mammal and pharmaceutical compositions containing such derivatives are disclosed.

### BACKGROUND OF THE INVENTION

Signal transduction, and the molecules associated with it, comprise a kind of biochemical language. In multicellular organisms, signal transduction messages act via receptors for extracellular biological signals such as growth factors or hormones to coordinate activities such as tissue growth and repair, stasis, and cell death. Medical science is still unraveling the details of many of the molecules and signal pathways in humans, but many genes and their respective proteins have been identified as agents of disease.

Uncontrolled signaling has been implicated in a variety of disease conditions including, inflammation, cancer, arteriosclerosis, and psoriasis. For example, many cancer causing genes (oncogenes) are protein kinases, enzymes that catalyze protein phosphorylation reactions, or are specifically regulated by phosphorylation. In addition, one kinase can have its activity regulated by one or more distinct protein kinases, resulting in specific signaling cascades.

The regulation of cell signaling events in the nucleus, for the coordinated control of target genes, allows cells to respond to external stimuli in a physiologically appropriate manner. Signal transduction pathways are crucial to the determination of the functional connections between transcriptional events regulated by nuclear steroid receptors and signaling cascades mediated by cell-surface growth factor receptors. The nuclear import of protein kinases provides one mechanism for modulating cellular signal transduction pathways.

Serum and glucocorticoid-inducible kinases (SGK) are a family of proteins in the serine/threonine protein kinase family. SGKs actively shuttle between the nucleus and the cytoplasm in synchrony with the cell cycle. SGK was originally identified as a glucocorticoid and osmotic stress-responsive gene.

It has been shown previously that SGK1 and protein kinase B (PKB) have similar specificities towards a panel of synthetic peptides, preferentially phosphorylating Ser and Thr residues that lie in Arg-Xaa-Arg-Xaa-Xaa-Ser/Thr motifs. The specificity requirements of SGK2 and SGK3 were also found to be similar, although SGK3 appears to tolerate the presence of a Lys instead of an Arg at position n-3 (where n is the site of phosphorylation) better than SGK1 or SGK2 (Kobayashi et al. (1999), Biochemical Journal, Vol. 344, pp. 189-197).

Because serum and glucocorticoid-induced protein kinases exert a regulatory effect on intracellular receptors by acting as transcriptional activators of genes, they play a fundamental role in the control of homeostasis, differentiation, and development in tissues. The utilization of phosphorylation-dephosphorylation networks of serine/threonine kinases by extracellular regulators, such as growth and differentiation factors, suggests that SGK2α protein kinase plays a role in the development and progression of diseases. SGK is activated by phosphorylation via other molecules when cells are stimulated by insulin, insulin-like growth factor-1, serum, or oxidative stress.

SGK proteins may be associated with cell survival (Mikosz et al. (2001) J. Biol. Chem. 276(20):16649-16654; and Brunet et al. (2001) Mol. Cell Biol. 21(3):952-965). Overexpression of SGK in cells protects cells from cell death after withdrawal of serum and growth factors, whereas overexpression of kinase-dead mutants fail to provide this protection. Moreover, overexpression of SGK in “normal” cells leads to increased cell proliferation.

### DESCRIPTION OF THE RELATED ART

U.S. Pat. No. 6,309,949 (Millennium Pharmaceuticals) describes methods of screening against SGK.

PCT Published Patent Application, WO 02/24947 (Kinetek Pharmaceuticals) describes cancer associated protein kinases and their uses.

U.S. Pat. No. 3,798,225 (Mead Johnson), U.S. Pat. No. 4,015,006 (Mead Johnson), U.S. Pat. No. 4,714,705 (Ortho Pharmaceutical), and U.S. Pat. No. 4,880,817 (Ortho) all describe isoquinolone derivatives useful as vasodilating agents.

### SUMMARY OF THE INVENTION

This invention is directed to the use of certain isoquinolone derivatives in treating hyperproliferative disorders, e.g., cancer, inflammation, etc. in a mammal. Of particular interest are hyperproliferative disorders associated with cellular modulation of protein phosphorylation states, i.e. altered activity of protein kinases. In one aspect of the invention, compounds and pharmaceutical compositions of the invention are used to inhibit the activity of SGK enzymes. Accordingly, in one aspect, this invention provides a method of treating cancer in a mammal, which method comprises administering to the mammal in need thereof a therapeutically effective amount of a compound of formula (I): 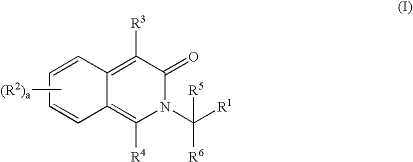
wherein:
a is 0 to 4;
R¹ is carbocyclyl or heterocyclyl;
each R² is selected from the group consisting of hydrogen, alkyl, alkenyl, aryl, aralkyl, aralkenyl, halo, haloalkyl, haloalkenyl, nitro, cyano, cycloalkyl, cycloalkylalkyl, cycloalkylalkenyl, heterocyclyl, heterocyclylalkyl, —OR⁷, —C(O)OR⁷, —C(O)N(R⁷)₂, —N(R⁷)₂, —N(R⁷)C(O)OR⁸, —N(R⁷)C(O)N(R⁷)₂, —N(R⁷)C(O)R⁷, —R⁹—N═N—O—R⁸, —S(O)ₚR⁷ (where p is 0 to 2), and —S(O)ₚN(R⁷)₂ (where p is 0 to 2);
R³ and R⁴ are each independently selected from the group consisting of hydrogen, alkyl, alkenyl, aryl, aralkyl, aralkenyl, halo, haloalkyl, haloalkenyl, nitro, cyano, cycloalkyl, cycloalkylalkyl, cycloalkylalkenyl, heterocyclyl, heterocyclylalkyl, —OR⁷, —C(O)OR⁷, —C(O)N(R⁷)₂, —N(R⁷)₂, —N(R⁷)C(O)OR⁸, —N(R⁷)C(O)N(R⁷)₂, —N(R⁷)C(O)R⁷, —R⁹—N═N—O—R⁸, —S(O)ₚR⁷ (where p is 0 to 2), and —S(O)ₚN(R⁷)₂ (where p is 0 to 2);
R⁵ and R⁶ are each independently selected from the group consisting of hydrogen, alkyl, alkenyl, haloalkyl, haloalkenyl, cycloalkyl, cycloalkenyl and heterocyclylalkyl;
each R⁷ is independently selected from the group consisting of hydrogen, alkyl, alkenyl, haloalkyl, haloalkenyl, aryl, aralkyl, aralkenyl, cycloalkyl, cycloalkylalkyl and cycloalkylalkenyl;
each R⁸ is independently selected from the group consisting of hydrogen, alkyl, alkenyl, haloalkyl, haloalkenyl, aralkyl, aralkenyl, cycloalkyl, cycloalkylalkyl and cycloalkylalkenyl; and
R⁹ is a bond or a straight or branched alkylene or alkenylene chain;
   as a single stereoisomer, a mixture of stereoisomers, or as a racemic mixture of stereoisomers; or as a solvate or polymorph; or as a pharmaceutically acceptable salt thereof.

In another aspect, this invention provides a method of treating hyperproliferative disorders in a mammal, which method comprises administering to the mammal in need thereof a therapeutically effective amount of a compound of formula (I), as set forth above, as a single stereoisomer, a mixture of stereoisomers, or as a racemic mixture of stereoisomers; or as a solvate or polymorph; or as a pharmaceutically acceptable salt thereof.

In another aspect, this invention provides a method of treating a mammal having a disorder or condition associated with hyperproliferation and tissue remodelling or repair, wherein said method comprises administering to the mammal having the disorder or condition a therapeutically effective amount of a compound of formula (I), as set forth above, as a single stereoisomer, a mixture of stereoisomers, or as a racemic mixture of stereoisomers; or as a solvate or polymorph; or as a pharmaceutically acceptable salt thereof.

In another aspect, this invention provides a method of treating a mammalian cell with a compound of formula (I), as set forth above, as a single stereoisomer, a mixture of stereoisomers, or as a racemic mixture of stereoisomers; or as a solvate or polymorph; or as a pharmaceutically acceptable salt thereof; wherein the method comprises administering the compound of formula (I) to a mammalian cell and the compound of formula (I) is capable of inhibiting the activity of SGK2 within the mammalian cell.

In another aspect, this invention provides a pharmaceutical composition useful in treating cancer or inflammation in a human, wherein the pharmaceutical composition comprises a pharmaceutically acceptable carrier, diluent or excipient and a compound of formula (I): 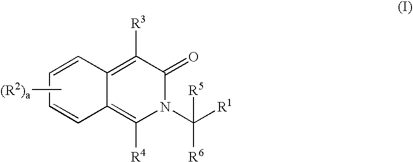
wherein:
a is 0 to 4;
R¹ is carbocyclyl or heterocyclyl;
each R² is selected from the group consisting of hydrogen, alkyl, alkenyl, aryl, aralkyl, aralkenyl, halo, haloalkyl, haloalkenyl, nitro, cyano, cycloalkyl, cycloalkylalkyl, cycloalkylalkenyl, heterocyclyl, heterocyclylalkyl, —OR⁷, —C(O)OR⁷, —C(O)N(R⁷)₂, —N(R⁷)₂, —N(R⁷)C(O)N(R⁷)₂, —N(R⁷)C(O)OR⁸, —N(R⁷)C(O)R⁷, —R⁹—N═N—O—R⁸, —S(O)ₚR⁷ (where p is 0 to 2), and —S(O)ₚN(R⁷)₂ (where p is 0 to 2);
R³ and R⁴ are each independently selected from the group consisting of hydrogen, alkyl, alkenyl, aryl, aralkyl, aralkenyl, halo, haloalkyl, haloalkenyl, nitro, cyano, cycloalkyl, cycloalkylalkyl, cycloalkylalkenyl, heterocyclyl, heterocyclylalkyl, —OR⁷, —C(O)OR⁷, —C(O)N(R⁷)₂, —N(R⁷)₂, —N(R⁷)C(O)OR⁸, —N(R⁷)C(O)N(R⁷)₂, —N(R⁷)C(O)R⁷, —R⁹—N═N—O—R⁸, —S(O)ₚR⁷ (where p is 0 to 2), and —S(O)ₚN(R⁷)₂ (where p is 0 to 2);
R⁵ and R⁶ are each independently selected from the group consisting of hydrogen, alkyl, alkenyl, haloalkyl, haloalkenyl, cycloalkyl, cycloalkenyl and heterocyclylalkyl;
each R⁷ is independently selected from the group consisting of hydrogen, alkyl, alkenyl, haloalkyl, haloalkenyl, aryl, aralkyl, aralkenyl, cycloalkyl, cycloalkylalkyl and cycloalkylalkenyl;
each R⁸ is independently selected from the group consisting of hydrogen, alkyl, alkenyl, haloalkyl, haloalkenyl, aralkyl, aralkenyl, cycloalkyl, cycloalkylalkyl and cycloalkylalkenyl; and
R⁹ is a bond or a straight or branched alkylene or alkenylene chain; as a single stereoisomer, a mixture of stereoisomers, or as a racemic mixture of stereoisomers; or as a solvate or polymorph; or as a pharmaceutically acceptable salt thereof, with the proviso that R¹ can not be unsubstituted phenyl when all of the following occur:

(i) a is 2 and one R² is methoxy in the 6-position of the isoquinolone ring and the other R² is methoxy in the 7-position of the isoquinolone ring; and

(ii) R³, R⁵ and R⁶ are all hydrogen, and (iii) R⁴ is 3,4-dimethoxybenzyl.

In another aspect of the invention, the use of the compounds of formula (I) for the treatment of cancer, inflammation, or disorders or condition associated with hyperproliferation and tissue remodelling or repair is provided.

In another aspect of the invention, the use of the compounds of formula (I) for the treatment of disorders associated with the activity of SGK are provided.

In another aspect, this invention provides compounds of formula (I) as set forth above for the pharmaceutical compositions of the invention, provided, however, that such compounds do not include compounds that are disclosed in J. Org. Chem. (1994), Vol. 59, No. 20, pp. 6116-6118, Heterocycles (1983), Vol. 20, No. 7, pp. 1367-1371, Heterocycles (1978), Vol. 9, No. 9, pp. 1197-1206, J. Med. Chem. (1972), Vol. 15, No. 11, pp. 1131-1135; or disclosed and/or claimed in Japanese Patent 54132583 (1978, Sankyo), U.S. Pat. No. 4,015,006 (Mead Johnson), U.S. Pat. No. 3,798,225 (Mead Johnson), U.S. Pat. No. 3,910,927 (Mead Johnson), U.S. Pat. No. 4,041,077 (UCB), U.S. Pat. No. 3,954,771 (UCB), and U.S. Pat. No. 3,872,130.

### DETAILED DESCRIPTION OF THE INVENTION

### Definitions

As used herein the singular forms “a”, “and”, and “the” include plural referents unless the context clearly dictates otherwise. For example, “a compound” refers to one or more of such compounds, while “the enzyme” includes a particular enzyme as well as other family members and equivalents thereof as known to those skilled in the art. As used in the specification and appended claims, unless specified to the contrary, the following terms have the meaning indicated.

“Alkyl” refers to a straight or branched hydrocarbon chain radical consisting solely of carbon and hydrogen atoms, containing no unsaturation, having from one to eight carbon atoms, and which is attached to the rest of the molecule by a single bond, e.g., methyl, ethyl, n-propyl, 1-methylethyl (iso-propyl), n-butyl, n-pentyl, 1,1-dimethylethyl (t-butyl), and the like. Unless stated otherwise specifically in the specification, the alkyl radical may be optionally substituted by one or more substituents selected from the group consisting of nitro, cyano, —OR⁷, —C(O)OR⁷, —C(O)N(R⁷)₂, —N(R⁷)₂, —N(R⁷)C(O)OR⁸, —N(R⁷)C(O)R⁷, —N(R⁷)C(O)N(R⁷)₂, —R⁹—N═N—O—R⁸, —S(O)ₚR⁷ (where p is 0 to 2), and —S(O)ₚN(R⁷)₂ (where p is 0 to 2) where each R⁷, R⁸ and R⁹ are as defined above in the Summary of the Invention. Unless stated otherwise specifically in the specification, it is understood that for radicals, as defined below, that contain a substituted alkyl group that the substitution can occur on any carbon of the alkyl group.

“Alkenyl” refers to a straight or branched hydrocarbon chain radical consisting solely of carbon and hydrogen atoms, containing at least one double bond, having from two to eight carbon atoms, and which is attached to the rest of the molecule by a single bond or a double bond, e.g., ethenyl, prop-1-enyl, but-1-enyl, pent-1-enyl, penta-1,4-dienyl, and the like. Unless stated otherwise specifically in the specification, the alkenyl radical may be optionally substituted by one or more substituents selected from the group consisting of nitro, cyano, —OR⁷, —C(O)OR⁷, —C(O)N(R⁷)₂, —N(R⁷)₂, —N(R⁷)C(O)OR⁸, —N(R⁷)C(O)R⁷, —N(R⁷)C(O)N(R⁷)₂, —R⁹—N═N—O—R⁸, —S(O)ₚR⁷ (where p is 0 to 2), and —S(O)ₚN(R⁷)₂ (where p is 0 to 2) where each R⁷, R⁸ and R⁹ are as defined above in the Summary of the Invention. Unless stated otherwise specifically in the specification, it is understood that for radicals, as defined below, that contain a substituted alkenyl group that the substitution can occur on any carbon of the alkenyl group.

“Aryl” refers to a phenyl or naphthyl radical. Unless stated otherwise specifically in the specification, the term “aryl” or the prefix “ar-” (such as in “aralkyl”) is meant to include aryl radicals optionally substituted by one or more substituents selected from the group consisting of nitro, cyano, cycloalkyl, cycloalkylalkyl, cycloalkylalkenyl, heterocyclyl, heterocyclylalkyl, —OR⁷, —C(O)OR⁷, —C(O)N(R⁷)₂, —N(R⁷)₂, —N(R⁷)C(O)OR⁸, —N(R⁷)C(O)N(R⁷)₂, —N(R⁷)C(O)R⁷, —R⁹—N═N—O—R⁸, —S(O)ₚR⁷ (where p is 0 to 2), and —S(O)ₚN(R⁷)₂ (where p is 0 to 2) where each R⁷, R⁸ and R⁹ are as defined above in the Summary of the Invention.

“Aralkyl” refers to a radical of the formula —RₐR_{b} where Rₐ is an alkyl radical as defined above and R_{b} is one or more aryl radicals as defined above, e.g., benzyl, diphenylmethyl and the like. The aryl radical(s) may be optionally substituted as described above.

“Aralkenyl” refers to a radical of the formula —R_{c}R_{b} where R_{c} is an alkenyl radical as defined above and R_{b} is one or more aryl radicals as defined above, e.g., 3-phenylprop-1-enyl, and the like. The aryl radical(s) and the alkenyl radical may be optionally substituted as described above.

“Alkylene” and “alkylene chain” refer to a straight or branched divalent hydrocarbon chain consisting solely of carbon and hydrogen, containing no unsaturation and having from one to eight carbon atoms, e.g., methylene, ethylene, propylene, n-butylene, and the like. The alkylene chain may be optionally substituted by one or more substituents selected from the group consisting of nitro, cyano, —OR⁷, —C(O)OR⁷, —C(O)N(R⁷)₂, —N(R⁷)₂, —N(R⁷)C(O)OR⁸, —N(R⁷)C(O)N(R⁷)₂, —N(R⁷)C(O)R⁷, —R⁹—N═N—O—R⁸, —S(O)ₚR⁷ (where p is 0 to 2), and —S(O)ₚN(R⁷)₂ (where p is 0 to 2) where each R⁷, R⁸ and R⁹ are as defined above in the Summary of the Invention. The alkylene chain may be attached to the rest of the molecule through any two carbons within the chain.

“Alkenylene chain” refers to a straight or branched divalent hydrocarbon chain consisting solely of carbon and hydrogen, containing at least one double bond and having from two to eight carbon atoms, e.g., ethenylene, prop-1-enylene, but-1-enylene, pent-1-enylene, hexa-1,4-dienylene, and the like. The alkenylene chain may be optionally substituted by one or more substituents selected from the group consisting of nitro, cyano, —OR⁷, —C(O)OR⁷, —C(O)N(R⁷)₂, —N(R⁷)₂, —N(R⁷)C(O)OR⁸, —N(R⁷)C(O)R⁷, —N(R⁷)C(O)N(R⁷)₂, —R⁹—N═N—O_{R}⁸, —S(O)ₚR⁷ (where p is 0 to 2), and —S(O)ₚN(R⁷)₂ (where p is 0 to 2) where each R⁷, R⁸ and R⁹ are as defined above in the Summary of the Invention. The alkenylene chain may be attached to the rest of the molecule through any two carbons within the chain.

“Carbocyclyl” refers to a stable 3- to 15-membered ring radical consisting solely of carbon and hydrogen atoms. For purposes of this invention, the carbocyclyl radical may be a monocyclic, bicyclic or tricyclic ring system, and may include fused or bridged ring systems, and the carbocyclyl may be partially or fully saturated or aromatic, and the carbon atoms in the carbocyclyl may be optionally oxidized. The carbocyclyl radical may be optionally substituted by one or more substituents selected from the group consisting of alkyl, alkenyl, aryl, aralkyl, aralkenyl, halo, haloalkyl, haloalkenyl, cycloalkyl, cycloalkylalkyl, heterocyclyl, heterocyclylalkyl, nitro, cyano, —OR⁷, —C(O)OR⁷, —C(O)N(R⁷)₂, —N(R⁷)₂, —N(R⁷)C(O)OR⁸, —N(R⁷)C(O)N(R⁷)₂, —N(R⁷)C(O)R⁷, —R⁹—N═N—O—R⁸, —S(O)ₚR⁷ (where p is 0 to 2), and —S(O)ₚN(R⁷)₂ (where p is 0 to 2) where each R⁷, R⁸ and R⁹ are as defined above in the Summary of the Invention. Examples of carbocyclyl radicals include, but are not limited to, cycloalkyl and aryl radicals as defined herein, and norbornane, norbornene, adamantyl, bicyclo[2.2.2]octane, indenyl8i, azulenyl, fluorenyl, anthracenyl, and the like.

“Cycloalkyl” refers to a stable monovalent monocyclic, bicyclic or tricyclic hydrocarbon radical consisting solely of carbon and hydrogen atoms, having from 3 to 15 carbon atoms, and which is saturated and attached to the rest of the molecule by a single bond, e.g., cyclopropyl, cyclobutyl, cyclopentyl, cyclohexyl, decalinyl and the like. Unless otherwise stated specifically in the specification, the term “cycloalkyl” is meant to include cycloalkyl radicals which are optionally substituted by one or more substituents independently selected from the group consisting of alkyl, alkenyl, aryl, aralkyl, aralkenyl, halo, haloalkyl, haloalkenyl, cycloalkyl, cycloalkylalkyl, heterocyclyl, heterocyclylalkyl, nitro, cyano, —OR⁷, —C(O)OR⁷, —C(O)N(R⁷)₂, —N(R⁷)₂, —N(R⁷)C(O)OR⁸, —N(R⁷)C(O)R⁷, —N(R⁷)C(O)N(R⁷)₂, —R⁹—N═N—O—R⁸, —S(O)ₚR⁷ (where p is 0 to 2), and —S(O)ₚN(R⁷)₂ (where p is 0 to 2) where each R⁷, R⁸ and R⁹ are as defined above in the Summary of the Invention.

“Cycloalkylalkyl” refers to a radical of the formula —RₐR_{d} where Rₐ is an alkyl radical as defined above and R_{d} is a cycloalkyl radical as defined above. The alkyl radical and the cycloalkyl radical may be optionally substituted as defined above.

“Cycloalkylalkenyl” refers to a radical of the formula —R_{f}R_{d} where R_{f} is an alkenyl radical as defined above and R_{d} is a cycloalkyl radical as defined above. The alkenyl radical and the cycloalkyl radical may be optionally substituted as defined above.

“Halo” refers to bromo, chloro, fluoro or iodo.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1-fluoromethyl-2-fluoroethyl, 3-bromo-2-fluoropropyl, 1-bromomethyl-2-bromoethyl, and the like.

“Haloalkenyl” refers to an alkenyl radical, as defined above, that is substituted by one or more halo radicals, as defined above, e.g., 2-ethenyl, 3-bromoprop-1-enyl, and the like.

“Haloalkoxy” refers to a radical of the formula —OR_{c} where R_{c} is an haloalkyl radical as defined above, e.g., trifluoromethoxy, difluoromethoxy, trichloromethoxy, 2,2,2-trifluoroethoxy, 1-fluoromethyl-2-fluoroethoxy, 3-bromo-2-fluoropropoxy, 1-bromomethyl-2-bromoethoxy, and the like.

“Heterocyclyl” refers to a stable 3- to 15-membered ring radical which consists of carbon atoms and from one to five heteroatoms selected from the group consisting of nitrogen, oxygen and sulfur. For purposes of this invention, the heterocyclyl radical may be a monocyclic, bicyclic or tricyclic ring system, which may include fused or bridged ring systems; and the nitrogen, carbon or sulfur atoms in the heterocyclyl radical may be optionally oxidized; the nitrogen atom may be optionally quaternized; and the heterocyclyl radical may be aromatic or partially or fully saturated. The heterocyclyl radical may not be attached to the rest of the molecule at any heteroatom atom. Examples of such heterocyclyl radicals include, but are not limited to, azepinyl, acridinyl, benzimidazolyl, benzthiazolyl, benzothiadiazolyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,5]imidazo[1,2-a]pyridinyl; carbazolyl, cinnolinyl, dioxolanyl, decahydroisoquinolyl, furanyl, furanonyl, isothiazolyl, imidazolyl, imidazolinyl, imidazolidinyl, isothiazolidinyl, indolyl, indazolyl, isoindolyl, indolinyl, isoindolinyl, indolizinyl, isoxazolyl, isoxazolidinyl, morpholinyl, naphthyridinyl, oxadiazolyl, octahydroindolyl, octahydroisoindolyl, 2-oxopiperazinyl, 2-oxopiperidinyl, 2-oxopyrrolidinyl, 2-oxoazepinyl, oxazolyl, oxazolidinyl, oxiranyl, piperidinyl, piperazinyl, 4-piperidonyl, phenazinyl, phenothiazinyl, phenoxazinyl, phthalazinyl, pteridinyl, purinyl, pyrrolyl, pyrrolidinyl, pyrazolyl, pyrazolidinyl, pyridinyl, pyrazinyl, pyrimidinyl, pyridazinyl, quinazolinyl, quinoxalinyl, quinolinyl, quinuclidinyl, isoquinolinyl, thiazolyl, thiazolidinyl, thiadiazolyl, triazolyl, tetrazolyl, tetrahydrofuryl, triazinyl, tetrahydropyranyl, thienyl, thiamorpholinyl, thiamorpholinyl sulfoxide, and thiamorpholinyl sulfone. Unless stated otherwise specifically in the specification, the term “heterocyclyl” is meant to include heterocyclyl radicals as defined above which are optionally substituted by one or more substituents selected from the group consisting of alkyl, alkenyl, aryl, aralkyl, aralkenyl, halo, haloalkyl, haloalkenyl, cycloalkyl, cycloalkylalkyl, heterocyclyl, heterocyclylalkyl, nitro, cyano, —OR⁷, —C(O)OR⁷, —C(O)N(R⁷)₂, —N(R⁷)₂, —N(R⁷)C(O)OR⁸, —N(R⁷)C(O)N(R⁷)₂, —N(R⁷)C(O)R⁷, —R⁹—N═N—O—R⁸, —S(O)ₚR⁷ (where p is 0 to 2), and —S(O)ₚN(R⁷)₂ (where p is 0 to 2) where each R⁷, R⁸ and R⁹ are as defined above in the Summary of the Invention.

“Heterocyclylalkyl” refers to a radical of the formula —RₐRₑ where Rₐ is an alkyl radical as defined above and Rₑ is a heterocyclyl radical as defined above, and if the heterocyclyl is a nitrogen-containing heterocyclyl, the heterocyclyl may be attached to the alkyl radical at the nitrogen atom. The heterocyclyl radical may be optionally substituted as defined above.

As used herein, compounds which are “commercially available” may be obtained from standard commercial sources including Acros Organics (Pittsburgh, Pa.), Aldrich Chemical (Milwaukee, Wis., including Sigma Chemical and Fluka), Apin Chemicals Ltd. (Milton Park, UK), Avocado Research (Lancashire, U.K.), BDH Inc. (Toronto, Canada), Bionet (Cornwall, U.K.), Chemservice Inc. (West Chester, Pa.), Crescent Chemical Co. (Hauppauge, N.Y.), Eastman Organic Chemicals, Eastman Kodak Company (Rochester, N.Y.), Fisher Scientific Co. (Pittsburgh, Pa.), Fisons Chemicals (Leicestershire, UK), Frontier Scientific (Logan, Utah), ICN Biomedicals, Inc. (Costa Mesa, Calif.), Key Organics (Cornwall, U.K.), Lancaster Synthesis (Windham, N.H.), Maybridge Chemical Co. Ltd. (Cornwall, U.K.), Parish Chemical Co. (Orem, Utah), Pfaltz & Bauer, Inc. (Waterbury, Conn.), Polyorganix (Houston, Tex.), Pierce Chemical Co. (Rockford, Ill.), Riedel de Haen A G (Hannover, Germany), Spectrum Quality Product, Inc. (New Brunswick, N.J.), TCI America (Portland, Oreg.), Trans World Chemicals, Inc. (Rockville, Md.), and Wako Chemicals USA, Inc. (Richmond, Va.).

As used herein, “suitable conditions” for carrying out a synthetic step are explicitly provided herein or may be discerned by reference to publications directed to methods used in synthetic organic chemistry. The reference books and treatise set forth above that detail the synthesis of reactants useful in the preparation of compounds of the present invention, will also provide suitable conditions for carrying out a synthetic step according to the present invention.

As used herein, “methods known to one of ordinary skill in the art” may be identified though various reference books and databases. Suitable reference books and treatise that describe the synthesis of reactants useful in the preparation of compounds of the present invention, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Specific and analogous reactants may also be identified through the indices of known chemicals prepared by the Chemical Abstract Service of the American Chemical Society, which are available in most public and university libraries, as well as through on-line databases (the American Chemical Society, Washington, D.C., www.acs.org may be contacted for more details). Chemicals that are known but not commercially available in catalogs may be prepared by custom chemical synthesis houses, where many of the standard chemical supply houses (e.g., those listed above) provide custom synthesis services.

“Prodrugs” is meant to indicate a compound that may be converted under physiological conditions or by solvolysis to a biologically active compound of the invention. Thus, the term “prodrug” refers to a metabolic precursor of a compound of the invention that is pharmaceutically acceptable. A prodrug may be inactive when administered to a subject in need thereof, but is converted in vivo to an active compound of the invention. Prodrugs are typically rapidly transformed in vivo to yield the parent compound of the invention, for example, by hydrolysis in blood. The prodrug compound often offers advantages of solubility, tissue compatibility or delayed release in a mammalian organism (see, Bundgard, H., Design of Prodrugs (1985), pp. 7-9, 21-24 (Elsevier, Amsterdam).

A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein.

The term “prodrug” is also meant to include any covalently bonded carriers which release the active compound of the invention in vivo when such prodrug is administered to a mammalian subject. Prodrugs of a compound of the invention may be prepared by modifying functional groups present in the compound of the invention in such a way that the modifications are cleaved, either in routine manipulation or in vivo, to the parent compound of the invention. Prodrugs include compounds of the invention wherein a hydroxy, amino or mercapto group is bonded to any group that, when the prodrug of the compound of the invention is administered to a mammalian subject, cleaves to form a free hydroxy, free amino or free mercapto group, respectively. Examples of prodrugs include, but are not limited to, acetate, formate and benzoate derivatives of alcohol and amine functional groups in the compounds of the invention and the like.

“Stable compound” and “stable structure” are meant to indicate a compound that is sufficiently robust to survive isolation to a useful degree of purity from a reaction mixture, and formulation into an efficacious therapeutic agent.

“Mammal” includes humans and domestic animals, such as cats, dogs, swine, cattle, sheep, goats, horses, rabbits, and the like.

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution.

“Pharmaceutically acceptable carrier, diluent or excipient” includes without limitation any adjuvant, carrier, excipient, glidant, sweetening agent, diluent, preservative, dye/colourant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid and the like, and organic acids such as acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminium salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyarine resins and the like. Particularly preferred organic bases are isopropylamine, diethylamine, ethanolamine, trimethylamine, dicyclohexylamine, choline and caffeine.

SGK refers to the Human Genome Organization (HUGO) Nomenclature Committee's name for serum and glucocorticoid-induced protein kinase. The sequence of SGK may be accessed at Genbank, NM_{—}005627, and is described by Waldegger et al. (1997) Proc. Natl. Acad. Sci. U.S.A. 94 (9), 4440-4445. The term SGK encompasses the different isoforms, namely SGK1, SGK2 and SGK3, as described by Kobayashi et al. (1999) Biochemical Journal 1999 344(1):189-97. In addition, there are two splice variants of SGK2; specifically, SGK2a and SGK2b. SGK2a encodes a protein of 367 residues with a calculated molecular mass of 41.1 kDa.

“Therapeutically effective amount” refers to that amount of a compound of formula (I) which, when administered to a mammal, preferably a human, is sufficient to effect treatment, as defined below, for cancer, inflammation, or neurological disease. The amount of a compound of formula (I) which constitutes a “therapeutically effective amount” will vary depending on the compound, the condition and its severity, and the age of the mammal to be treated, but can be determined routinely by one of ordinary skill in the art having regard to his own knowledge and to this disclosure.

“Treating” or “treatment” as used herein covers the treatment of disease as disclosed herein, in a mammal, preferably a human, and includes:

(i) preventing cancer or inflammation from occurring in a mammal, in particular, when such mammal is predisposed to the condition but has not yet been diagnosed as having it;

(ii) inhibiting cancer or inflammation, i.e., arresting its development; or

(iii) relieving cancer or inflammation, i.e., causing regression of the condition.

The compounds of formula (I), or their pharmaceutically acceptable salts may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present invention is meant to include all such possible isomers, as well as, their racemic and optically pure forms. Optically active (+) and (−), (R)- and (S)-, or (D)- and (L)-isomers may be prepared using chiral synthons or chiral reagents, or resolved using conventional techniques, such as reverse phase HPLC. When the compounds described herein contain olefinic double bonds or other centers of geometric asymmetry, and unless specified otherwise, it is intended that the compounds include both E and Z geometric isomers. Likewise, all tautomeric forms are also intended to be included.

The nomenclature used herein for the compounds of formula (I) is a modified form of the I.U.P.A.C. nomenclature system wherein the compounds are named herein as derivatives of the isoquinolone moiety.

### Methods of Use

This invention is directed to methods of using compounds of formula (I), as set forth above in the Summary of the Invention, and pharmaceutical compositions containing compounds of formula (I) in treating hyperproliferative conditions. Thus, the methods disclosed herein are useful in treating disorders and physiological conditions associated with hyperproliferation and tissue remodelling or repair when administered to a subject in need of such treatment. Of particular interest are hyperproliferative disorders associated with cellular modulation of protein phosphorylation states, i.e. altered activity of phosphorylation modifying enzyme(s), e.g. protein kinases.

In one aspect of the invention, compounds and pharmaceutical compositions of the invention are used to inhibit the activity of SGK. Levels of SGK mRNA are increased or affected by a number of diverse stimuli, including: exposure to follicle-stimulating hormone; high extracellular osmolarity; injury of the brain; and aldosterone. The levels of RNA expression of SGK are elevated 2-3 fold in liver and lung tumour tissue compared to control tissue. Immunohistochemical analysis of colon cancer shows elevation of SGK2 in the cytoplasm and SGK2 RNA expression levels are elevated in colon (LS-180 and HT-29) and prostate (LnCaP, DU-145) cancer cell lines as well as a NSCLC cell line (A549). In contrast, expression levels of SGK2 in “normal” cell lines are very low.

The compounds and pharmaceutical compositions of the invention are administered to a subject having a cancer or a pathological inflammation in order to inhibit tumour growth by impeding cell division, and to decrease inflammation by inhibiting cell adhesion and cell migration.

The methods of the invention can be used prophylactically (i.e., to prevent the disorder of interest from occurring) or therapeutically (i.e., to inhibit or relieve the disorder). As used herein, the term “treating” is used to refer to both prevention of disease, and treatment of pre-existing conditions. The prevention of symptoms is accomplished by administration of the compounds and pharmaceutical compositions of the invention prior to development of overt disease, e.g., to prevent the regrowth of tumours, prevent metastatic growth, diminish restenosis associated with cardiovascular surgery, to prevent or reduce cell migration leading to inflammation and associated tissue damage. Alternatively, the compounds and pharmaceutical compositions of the invention may be administered to a subject in need thereof to treat an ongoing disease, by stabilizing or improving the clinical symptoms of the patient.

The subject, or patient, may be from any mammalian species, e.g. primates, particularly humans; rodents, including mice, rats and hamsters; rabbits; equines; bovines; canines; felines; etc. Animal models are of interest for experimental investigations, providing a model for treatment of human disease.

Hyperproliferative disorders refers to excess cell proliferation, relative to that occurring with the same type of cell in the general population and/or the same type of cell obtained from a patient at an earlier time. The term denotes malignant as well as non-malignant cell populations. Such disorders have an excess cell proliferation of one or more subsets of cells, which often appear to differ from the surrounding tissue both morphologically and genotypically. The excess cell proliferation can be determined by reference to the general population and/or by reference to a particular patient, e.g. at an earlier point in the patient's life. Hyperproliferative cell disorders can occur in different types of animals and in humans, and produce different physical manifestations depending upon the affected cells.

Hyperproliferative cell disorders include cancers; blood vessel proliferative disorders such as restenosis, atherosclerosis, in-stent stenosis, vascular graft restenosis, etc.; fibrotic disorders; inflammatory disorders, e.g. arthritis, etc.; endometriosis; benign growth disorders such as prostate enlargement and lipomas; and autoimmune disorders. Cancers of particular interest include carcinomas, e.g. colon, prostate, breast, melanoma, ductal, endometrial, stomach, dysplastic oral mucosa, invasive oral cancer, non-small cell lung carcinoma, transitional and squamous cell urinary carcinoma, etc.; neurological malignancies, e.g. neuroblastoma, gliomas, etc.; hematological malignancies, e.g. childhood acute leukaemia, non-Hodgkin's lymphomas, chronic lymphocytic leukaemia, malignant cutaneous T-cells, mycosis fungoides, non-MF cutaneous T-cell lymphoma, lymphomatoid papulosis, T-cell rich cutaneous lymphoid hyperplasia, bullous pemphigoid, discoid lupus erythematosus, lichen planus, etc.; sarcomas, melanomas, adenomas; benign lesions such as papillomas, and the like.

Other hyperproliferative disorders that may be associated with altered activity of phosphorylation modifying enzyme(s) include a variety of conditions where there is proliferation and/or migration of smooth muscle cells, and/or inflammatory cells into the intimal layer of a vessel, resulting in restricted blood flow through that vessel, i.e. neointimal occlusive lesions. Occlusive vascular conditions of interest include atherosclerosis, graft coronary vascular disease after transplantation, vein graft stenosis, peri-anastomatic prosthetic graft stenosis, restenosis after angioplasty or stent placement, and the like.

Disorders and conditions where there is hyperproliferation and/or tissue remodelling or repair of reproductive tissue, e.g. uterine, testicular and ovarian carcinomas, endometriosis, squamous and glandular epithelial carcinomas of the cervix, etc. are reduced in cell number by administration of the compounds and pharmaceutical compositions of the invention. Other disorders and conditions of interest relate to epidermal hyperproliferation, tissue remodelling and repair. For example, the chronic skin inflammation of psoriasis is associated with hyperplastic epidermal keratinocytes.

Other disorders of interest include inflammatory disorders and autoimmune conditions including, but not limited to, psoriasis, rheumatoid arthritis, multiple sclerosis, scleroderma, systemic lupus erythematosus, Sjögren's syndrome, atopic dermatitis, asthma, and allergy. Target cells susceptible to the treatment include cells involved in instigating autoimmune reactions as well as those suffering or responding from the effects of autoimmune attack or inflammatory events, and include lymphocytes and fibroblasts.

The susceptibility of a particular cell to treatment according to the invention may be determined by in vitro testing. Typically, a culture of the cell is combined with a subject compound at varying concentrations for a period of time sufficient to allow the active agents to induce cell death or inhibit migration, usually between about one hour and one week. For in vitro testing, cultured cells from a biopsy sample may be used.

The dose will vary depending on mode of administration, specific disorder, patient status, etc. Typically a therapeutic dose will be sufficient to substantially decrease the undesirable cell population in the targeted tissue, while maintaining patient viability. Treatment will generally be continued until there is a substantial reduction, e.g. at least about 50%, decrease in the clinical manifestation of disease, and may be continued until there are essentially none of the undesirable cellular activity detected in the relevant tissue.

A protein kinase of particular interest is the serum and glucocorticoid-induced protein kinase (SGK) family, SGK1, SGK2a, SGK2b and SGK3. Dot blot analysis of probes prepared from mRNA of tumours showed that expression of SGK is consistently up-regulated in human tumour tissue. SGK can activate downstream targets of the P13 kinase pathway as shown in PCT Published Patent Application, WO 02/24947. SGKs actively shuttle between the nucleus and the cytoplasm in synchrony with the cell cycle. There is markedly enhanced transcription of SGK gene in diabetic nephropathy, with particularly high expression in mesangial cells, interstitial cells, and cells in the thick ascending limbs of the loop of Henle and distal tubules (Lang, F. et al., Proc. Nat. Acad. Sci. (2000), Vol. 97, pp. 8157-8162). The enhanced SGK transcription, which results from excessive extracellular glucose concentrations, stimulates renal tubular Na(+) transport.

In one embodiment of the invention, methods are provided for using compounds of formula (I) and pharmaceutical compositions containing such compounds in treating hyperproliferative disorders. Thus, the methods disclosed herein are useful in treating disorders and physiological conditions associated with hyperproliferation and tissue remodeling or repair when administered to a subject in need of such treatment. The compounds and pharmaceutical compositions of the invention are administered to a subject having a cancer or a pathological inflammation in order to inhibit tumour growth by impeding cell division, and to decrease inflammation by inhibiting cell adhesion and cell migration. The compounds of formula (I) may also find use as affinity reagents for the isolation and/or purification of SGKs using the biochemical affinity of the enzyme for inhibitors that act on it. The compounds are coupled to a matrix or gel. The coupled support is then used to separate the enzyme, which binds to the compound, from a sample mixture, e.g., a cell lysate, which may be optionally partially purified. The sample mixture is contacted with the compound-coupled support under conditions that minimize non-specific binding. Methods known in the art include columns, gels, capillaries, etc. The unbound proteins are washed free of the resin and the bound proteins are then eluted in a suitable buffer.

The compounds of formula (I) are also useful as reagents for studying signal transduction or any of the clinical disorders listed throughout this application, and for use as a positive control in high throughput screening.

### Administration of the Compounds and Pharmaceutical Compositions of the Invention

Administration of the compounds of the invention, or their pharmaceutically acceptable salts, in pure form or in an appropriate pharmaceutical composition, can be carried out via any of the accepted modes of administration of agents for serving similar utilities. The pharmaceutical compositions of the invention can be prepared by combining a compound of the invention with an appropriate pharmaceutically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. Typical routes of administering such pharmaceutical compositions include, without limitation, oral, topical, transdermal, inhalation, parenteral, sublingual, rectal, vaginal, and intranasal. The term parenteral as used herein includes subcutaneous injections, intravenous, intramuscular, intrasternal injection or infusion techniques. Pharmaceutical compositions of the invention are formulated so as to allow the active ingredients contained therein to be bioavailable upon administration of the composition to a patient. Compositions that will be administered to a subject or patient take the form of one or more dosage units, where for example, a tablet may be a single dosage unit, and a container of a compound of the invention in aerosol form may hold a plurality of dosage units. Actual methods of preparing such dosage forms are known, or will be apparent, to those skilled in this art; for example, see Remington's Pharmaceutical Sciences, 18th Ed., (Mack Publishing Company, Easton, Pa., 1990). The composition to be administered will, in any event, contain a therapeutically effective amount of a compound of the invention, or a pharmaceutically acceptable salt thereof, for treatment of a disorder or condition associated with hyperproliferation and tissue remodelling or repair in accordance with the teachings of this invention.

A pharmaceutical composition of the invention may be in the form of a solid or liquid. In one aspect, the carrier(s) are particulate, so that the compositions are, for example, in tablet or powder form. The carrier(s) may be liquid, with the compositions being, for example, an oral syrup, injectable liquid or an aerosol, which is useful in, e.g., inhalatory administration.

When intended for oral administration, the pharmaceutical composition is preferably in either solid or liquid form, where semi-solid, semi-liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like form. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel™, corn starch and the like; lubricants such as magnesium stearate or Sterotex™; glidants such as colloidal silicon dioxide; sweetening agents such as sucrose or saccharin; a flavoring agent such as peppermint, methyl salicylate or orange flavoring; and a colouring agent.

When the pharmaceutical composition is in the form of a capsule, e.g., a gelatin capsule, it may contain, in addition to materials of the above type, a liquid carrier such as polyethylene glycol or oil.

The pharmaceutical composition may be in the form of a liquid, e.g., an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colourant and flavor enhancer. In a composition intended to be administered by injection, one or more of a surfactant, preservative, wetting agent, dispersing agent, suspending agent, buffer, stabilizer and isotonic agent may be included.

The liquid pharmaceutical compositions of the invention, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid pharmaceutical composition of the invention intended for either parenteral or oral administration should contain an amount of a compound of the invention such that a suitable dosage will be obtained. Typically, this amount is at least 0.01% of a compound of the invention in the composition. When intended for oral administration, this amount may be varied to be between 0.1 and about 70% of the weight of the composition. Preferred oral pharmaceutical compositions contain between about 4% and about 50% of the compound of the invention. Preferred pharmaceutical compositions and preparations according to the present invention are prepared so that a parenteral dosage unit contains between 0.01 to 1% by weight of the compound of the invention.

The pharmaceutical composition of the invention may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device. Topical formulations may contain a concentration of the compound of the invention from about 0.1 to about 10% w/v (weight per unit volume).

The pharmaceutical composition of the invention may be intended for rectal administration, in the form, e.g., of a suppository, which will melt in the rectum and release the drug. The composition for rectal administration may contain an oleaginous base as a suitable nonirritating excipient. Such bases include, without limitation, lanolin, cocoa butter and polyethylene glycol.

The pharmaceutical composition of the invention may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule.

The pharmaceutical composition of the invention in solid or liquid form may include an agent that binds to the compound of the invention and thereby assists in the delivery of the compound. Suitable agents that may act in this capacity include a monoclonal or polyclonal antibody, a protein or a liposome.

The pharmaceutical composition of the invention may consist of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols of compounds of the invention may be delivered in single phase, bi-phasic, or tri-phasic systems in order to deliver the active ingredient(s). Delivery of the aerosol includes the necessary container, activators, valves, subcontainers, and the like, which together may form a kit. One skilled in the art, without undue experimentation may determine preferred aerosols.

Whether in solid, liquid or gaseous form, the pharmaceutical composition of the present invention may contain one or more known pharmacological agents used in the treatment of cancer or inflammation in a mammal, particularly, cancer or inflammation associated with hyperproliferation and tissue remodelling or repair.

The pharmaceutical compositions of the invention may be prepared by methodology well known in the pharmaceutical art. For example, a pharmaceutical composition intended to be administered by injection can be prepared by combining a compound of the invention with sterile, distilled water so as to form a solution. A surfactant may be added to facilitate the formation of a homogeneous solution or suspension. Surfactants are compounds that non-covalently interact with the compound of the invention so as to facilitate dissolution or homogeneous suspension of the compound in the aqueous delivery system.

The compounds of the invention, or their pharmaceutically acceptable salts, are administered in a therapeutically effective amount, which will vary depending upon a variety of factors including the activity of the specific compound employed; the metabolic stability and length of action of the compound; the age, body weight, general health, sex, and diet of the patient; the mode and time of administration; the rate of excretion; the drug combination; the severity of the particular disorder or condition; and the subject undergoing therapy. Generally, a therapeutically effective daily dose is from about 0.1 mg to about 20 mg/kg of body weight per day of a compound of the invention, or a pharmaceutically acceptable salt thereof; preferably, from about 0.1 mg to about 10 mg/kg of body weight per day; and most preferably, from about 0.1 mg to about 7.5 mg/kg of body weight per day.

### Preferred Embodiments of the Invention

Of the various methods of treating cancer or inflammation in a mammal as set forth above in the Summary of the Invention, a preferred method is that method wherein the cancer or inflammation is associated with hyperproliferation or cell survival. Another preferred method is that method wherein the cancer or inflammation is associated with the activity SGK.

Of the various methods of treating a mammalian cell with a compound of formula (I) as set forth above in the Summary of the Invention wherein the method comprises administering the compound of formula (I) to a mammalian cell and the compound of formula (I) is capable of inhibiting the activity of SGK within the mammalian cell in vitro. An alternate preferred method is that method wherein the mammalian cell is treated in vivo. Another preferred method is that method wherein the inhibition of activity results in a reduction of cell division or cell survival or overall tumour growth.

Of the various methods of treating a mammal as set forth above in the Summary of the Invention, a preferred method is that method wherein the mammal is a human.

Of the various methods or pharmaceutical compositions and the preferred methods or pharmaceutical compositions set forth herein and above in the Summary of the Invention, a preferred method or pharmaceutical composition is wherein the compound of formula (I) is a compound of formula (I) where R¹ is carbocyclyl.

Of this preferred method or pharmaceutical composition a more preferred method or pharmaceutical composition is that method or pharmaceutical composition wherein the compound of formula (I) is a compound of formula (I) where R¹ is aryl.

Another more preferred method or pharmaceutical composition is that method or pharmaceutical composition wherein the compound of formula (I) is a compound of formula (I) where R¹ is cycloalkyl.

Another preferred method or pharmaceutical composition is wherein the compound of formula (I) is a compound of formula (I) where R¹ is heterocyclyl.

Of the various methods or pharmaceutical compositions and the preferred methods or pharmaceutical compositions set forth herein and above in the Summary of the Invention, another preferred method or pharmaceutical composition is wherein the compound of formula (I) is a compound of formula (I) where at least one R² is hydrogen, alkyl, alkenyl, cycloalkyl, cycloalkylalkyl or cycloalkylalkenyl.

Another preferred method or pharmaceutical composition is wherein the compound of formula (I) is a compound of formula (I) where at least one R² is aryl, aralkyl or aralkenyl.

Another preferred method or pharmaceutical composition is wherein the compound of formula (I) is a compound of formula (I) where at least one R² is halo, haloalkyl or haloalkenyl.

Another preferred method or pharmaceutical composition is wherein the compound of formula (I) is a compound of formula (I) where at least one R² is nitro, cyano, —N(R⁷)₂, —N(R⁷)C(O)OR⁸, —N(R⁷)C(O)R⁷ or —R⁹—N═N—O—R⁸.

Another preferred method or pharmaceutical composition is wherein the compound of formula (I) is a compound of formula (I) where at least one R² is heterocyclyl or heterocyclylalkyl.

Another preferred method or pharmaceutical composition is wherein the compound of formula (I) is a compound of formula (I) where at least one R² is —C(O)OR⁷ or —C(O)N(R⁷)₂.

Another preferred method or pharmaceutical composition is wherein the compound of formula (I) is a compound of formula (I) where at least one R² is —OR⁷, —S(O)ₚR⁷ (where p is 0 to 2), or —S(O)ₚN(R⁷)₂ (where p is 0 to 2).

Of the various methods or pharmaceutical compositions and the preferred methods or pharmaceutical compositions set forth herein and above in the Summary of the Invention, another preferred method or pharmaceutical composition is wherein the compound of formula (I) is a compound of formula (I) where R³ is hydrogen, alkyl, alkenyl, halo, haloalkyl, haloalkenyl, cycloalkyl, cycloalkylalkyl or cycloalkylalkenyl.

Another preferred method or pharmaceutical composition is wherein the compound of formula (I) is a compound of formula (I) where R³ is aryl, aralkyl or aralkenyl.

Another preferred method or pharmaceutical composition is wherein the compound of formula (I) is a compound of formula (I) where R³ is nitro, cyano, —N(R⁷)₂, —N(R⁷)C(O)OR⁸, —N(R⁷)C(O)R⁷ or —R⁹—N═N—O—R⁸.

Another preferred method or pharmaceutical composition is wherein the compound of formula (I) is a compound of formula (I) where R³ is heterocyclyl or heterocyclylalkyl.

Another preferred method or pharmaceutical composition is wherein the compound of formula (I) is a compound of formula (I) where R³ is —C(O)OR⁷ or —C(O)N(R⁷)₂.

Another preferred method or pharmaceutical composition is wherein the compound of formula (I) is a compound of formula (I) where R³ is —OR⁷, —S(O)ₚR⁷ (where p is 0 to 2) or —S(O)ₚN(R⁷)₂ (where p is 0 to 2).

Of the various methods or pharmaceutical compositions and the preferred methods or pharmaceutical compositions set forth herein and above in the Summary of the Invention, another preferred method or pharmaceutical composition is wherein the compound of formula (I) is a compound of formula (I) where R⁴ is hydrogen, alkyl, alkenyl, halo, haloalkyl, haloalkenyl, cycloalkyl, cycloalkylalkyl or cycloalkylalkenyl.

Another preferred method or pharmaceutical composition is wherein the compound of formula (I) is a compound of formula (I) where R⁴ is aryl, aralkyl or aralkenyl.

Another preferred method or pharmaceutical composition is wherein the compound of formula (I) is a compound of formula (I) where R⁴ is nitro, cyano, —N(R⁷)₂, —N(R⁷)C(O)OR⁸, —N(R⁷)C(O)R⁷ or —R⁹—N═N—O—R⁸.

Another preferred method or pharmaceutical composition is wherein the compound of formula (I) is a compound of formula (I) where R⁴ is heterocyclyl or heterocyclylalkyl.

Another preferred method or pharmaceutical composition is wherein the compound of formula (I) is a compound of formula (I) where R⁴ is —C(O)OR⁷ or —C(O)N(R⁷)₂.

Another preferred method or pharmaceutical composition is wherein the compound of formula (I) is a compound of formula (I) where R⁴ is —OR⁷, —S(O)ₚR⁷ (where p is 0 to 2) or —S(O)ₚN(R⁷)₂ (where p is 0 to 2).

Of the various methods or pharmaceutical compositions and the preferred methods or pharmaceutical compositions set forth herein and above in the Summary of the Invention, another preferred method or pharmaceutical composition are those wherein the compound of formula (I) is a compound of formula (I) where R⁵ and R⁶ are each independently selected from the group consisting of hydrogen, alkyl or haloalkyl.

### Preparation of the Compounds of Formula (I)

Compounds of formula (I) in the methods and pharmaceutical compositions of the invention may be prepared according to methods known to one skilled in the art, or by the methods similar to those disclosed in U.S. Pat. No. 3,798,225 (Mead Johnson), U.S. Pat. No. 4,015,006 (Mead Johnson), U.S. Pat. No. 4,041,077 (UCB, Societé Anonyme), U.S. Pat. No. 4,714,705 (Ortho Pharmaceutical), and U.S. Pat. No. 4,880,817 (Ortho Pharmaceutical) (all of which are incorporated in full by reference herein), or by methods similar to the method described below.

It is understood that in the following description, combinations of substituents and/or variables of the depicted formulae are permissible only if such contributions result in stable compounds.

It will also be appreciated by those skilled in the art that in the process described below the functional groups of intermediate compounds may need to be protected by suitable protecting groups. Such functional groups include hydroxy, amino, mercapto and carboxylic acid. Suitable protecting groups for hydroxy include trialkylsilyl or diarylalkylsilyl (e.g., t-butyldimethylsilyl, t-butyldiphenylsilyl or trimethylsilyl), tetrahydropyranyl, benzyl, and the like. Suitable protecting groups for amino, amidino and guanidino include t-butoxycarbonyl, benzyloxycarbonyl, and the like. Suitable protecting groups for mercapto include —C(O)—R (where R is alkyl, aryl or aralkyl), p-methoxybenzyl, trityl and the like. Suitable protecting groups for carboxylic acid include alkyl, aryl or aralkyl esters.

Protecting groups may be added or removed in accordance with standard techniques, which are well-known to those skilled in the art and as described herein.

The use of protecting groups is described in detail in Green, T. W. and P. G. M. Wutz, Protective Groups in Organic Synthesis (1991), 2nd Ed., Wiley-Interscience. The protecting group may also be a polymer resin such as a Wang resin or a 2-chlorotrityl chloride resin.

It will also be appreciated by those skilled in the art, although such protected derivatives of compounds of formulae (I), as described above in the Summary of the Invention, may not possess pharmacological activity as such, they may be administered to a mammal with cancer or inflammation and thereafter metabolized in the body to form compounds of the invention which are pharmacologically active. Such derivatives may therefore be described as “prodrugs”. All prodrugs of compounds of formula (I) are included within the scope of the invention.

The following Reaction Scheme illustrates a method to make compounds of formula (I). In this Reaction Scheme, R^{1a} is a radical of the formula —C(R⁵)(R⁶)R¹, and a, R¹, R², R³, R⁴, R⁵ and R⁶ are as defined in the Summary of the Invention for compounds of formula (I), and X is halo.

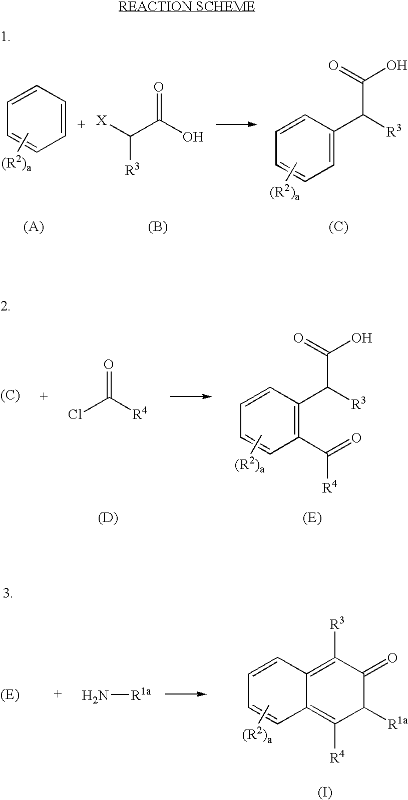

In this general scheme, starting components may be obtained from sources such as Aldrich, or synthesized according to sources known to those of ordinary skill in the art (see, e.g., Smith and March, March's Advanced Organic Chemistry: Reactions, Mechanisms, and Structure, 5th edition (Wiley Interscience, New York)). Moreover, groups R^{1a} through R⁴ are selected from components as indicated in the specification heretofore, and may be attached to starting components, intermediate components, and/or final products according to schemes known to those of ordinary skill in the art (see, e.g., U.S. Pat. No. 3,798,225 and U.S. Pat. No. 4,880,817).

An optionally-substituted phenylacetic acid of formula (C) can be either obtained from any chemical suppliers, such as Aldrich, or prepared under standard electrophilic aromatic substitution conditions according to schemes known to those of ordinary skill in the art (see, e.g., Smith and March, March's Advanced Organic Chemistry: Reactions, Mechanisms, and Structure, 5th edition (Wiley Interscience, New York)). As one example, a compound of formula (C) is formed according to Step 1 of the Reaction Scheme depicted herein, whereby an optionally-substituted benzene of formula (A) is combined with an optionally-substituted α-haloacetic acid of formula (B) (wherein X is halo) in the presence of a catalyst such as an aluminium halide salt, with optional heating and/or stirring.

A carbonyl-substituted phenylacetic acid of formula (E) may be prepared under standard electrophilic aromatic substitution conditions known to those of ordinary skill in the art (see, e.g., Smith and March, March's Advanced Organic Chemistry: Reactions, Mechanisms, and Structure, 5th edition (Wiley Interscience, New York)). In one example, a formylated phenylacetic acid of formula (E) (wherein R⁴ is hydrogen) may be prepared according to Step 2 of the Reaction Scheme depicted herein, by combining an optionally-substituted phenylacetic acid of formula (C) with zinc cyanide, hydrochloric acid, and water, under general Gatterman reaction conditions. In another example, a ketone-substituted phenylacetic acid of formula (E) (wherein R⁴ is optionally-substituted hydrocarbyl as indicated in the specification heretofore) may be prepared according to Step 2 of the Reaction Scheme depicted herein, by combining an optionally-substituted phenylacetic acid of formula (C) with an acyl halide in the presence of a catalyst such as an aluminium halide salt, with optional heating and/or stirring. Treatments such as those described herein may afford compounds of formula (E) and/or compound derivatives of formula (E) wherein the ring-substituted moieties are present in ring positions different to those disclosed herein, or are present in ring positions in addition to those disclosed herein. Such variability in the reaction product mixture may possibly be avoided by modifying reaction conditions according to schemes known to those of ordinary skill in the art. Moreover, the product components of a reaction product mixture may be separated from each other and purified through the use of a preparative separation and isolation technique such as high performance liquid chromatography (HPLC).

An N-substituted pyridinone compound of formula (I) can be prepared under standard cyclization conditions according to schemes known to those of ordinary skill in the art (see, e.g., Smith and March, March's Advanced Organic Chemistry: Reactions, Mechanisms, and Structure, 5th edition (Wiley Interscience, New York); U.S. Pat. No. 3,798,225). As one example, according to Step 3 of the Reaction Scheme depicted herein, a carbonyl-substituted phenylacetic acid of formula (E) is combined with an alkylamine acetate and acetic acid, to afford an N-alkyl pyridinone compound of formula (I).

### EXPERIMENTAL

### Example 1

### SGK Preparation and Assay

A. Enzyme Preparation:

SGK2α cDNA was cloned from human brain cDNA library by the polymerase chain reaction (PCR) technique and subsequently inserted directly into pPCR-2.1 T/A cloning vector (Strategene). After DNA sequencing, the confirmed wild-type human SGK2α cDNA was subcloned into baculovirus expression vector pAcG2T (BD PharMingen) for recombinant protein expression in insect cells. Expression of human SGK2α as a fusion protein required that the cDNA be ligated into the polyclonal site situated in frame and downstream of the glutathione-S-transferase gene of the baculovirus transfer vector pAcG2T. The 1104 base pair SGK2α coding sequence was excised from the pPCR-2.1 T/A vector using the Bam HI and Eco RI restriction sites generated the 5′ and 3′ of the coding sequence respectively by PCR amplification. This DNA fragment was directionally subcloned in-frame into the Bam HI and Eco RI polyclonal sites of the pAcG2T vector. Positive recombinant clones of SGK2α in the pAcG2T vector were determined by restriction enzyme analysis with Bam HI and Eco RI restriction enzymes. Sequence analysis of the recombinant clone revealed no discordance with the original wild type sequence.

Active SGK2α enzyme was expressed using the baculovirus expression vector system in a two-step process. In the first step, the infectious baculovirus particles were amplified prior to the recombinant overexpression of the protein. Infectious baculovirus was generated by co-transfecting recombinant pAcG2T-SGK2α plasmid with linear AcNPV (Autographa californica nuclear polyhedrosis virus (BD PharMingen), DNA into adherent Spodoptera frugiperda Sf9 insect cells (Invitrogen) following the manufacturer's instructions. Recombination between homologous sites allowed the heterologous SGK2α gene transfer from the transfer vector pAcG2T-SGK2α to the genomic AcNPV DNA and finally the production and amplification of packaged baculovirus particles. Expression of recombinant GST-SGK2α was under the control of the very late polyhedrin promoter activated after virion assembly was complete. After about 72 hour expression in High Five™ cells (Invitrogen), the recombinant GST-SGK2α protein was activated in vivo with 100 nM okadaic (Sigma) acid for three hours before cell harvesting. The addition of the phosphatase inhibitor, okadaic acid, effectively increased the phosphorylation and activation of the expressed SGK2α enzyme within the insect cells during the final stages of protein expression in Sf9 cells by inhibiting the activity of protein phosphatases like PP2A. The cell pellet was lysed by mild sonication in the lysis buffer (50 mM Tris-HCl, pH7.5, 2.5 mM EDTA, 150 mM NaCl, 1% NP-40, 200 nM okadaic acid, 0.1% β-mercaptoethanol, 0.5 mM sodium orthovanadate, 50 mM β-glycerophosphate, 1 mM NaF, 1 mM benzamidine and 0.5% (V/V) protease inhibitor cocktail set III (CalBiochem)). The lysate was cleared of cellular debris by centrifugation. The active recombinant protein was purified from the supernatant using a GST-glutathione affinity system according the manufacturer's instructions (Pharmacia). Following batch binding of the fusion protein to the glutathione-agarose beads, the matrix was transferred to a 1×10 cm Flex-column™ (Kontes Glass) chromatography system. The column was washed with high-salt buffer (50 mM Tris-HCl, pH7.5, 1 mM EDTA, 500 mM NaCl, 0.1% NP-40, 0.1% β-mercaptoethanol, 0.5 mM sodium orthovanadate, 50 mM β-glycerophosphate, 1 mM NaF, 1 mM benzamidine and 0.1 mM PMSF), then low-salt buffer (50 mM Tris-HCl, pH7.5, 1 mM EDTA, 50 mM NaCl, 0.1% β-mercaptoethanol, 0.5 mM sodium orthovanadate, 50 mM β-glycerophosphate, 1 mM benzamidine and 0.1 mM PMSF). The GST-SGK2α fusion protein was then released from the matrix using a glutathione buffer (50 mM Tris-HCl, pH7.5, 50 mM NaCl, 10 mM glutathione, 0.1% β-mercaptoethanol and 0.1 mM PMSF). A complete quantitative and qualitative analysis of the protein was monitored using Coomassie blue staining and Western blot analysis using anti-GST specific antibody (Kinetek). SGK2α protein comprised approximately 5% to 7% of the total insect cellular protein concentration.

Biochemical analysis of the activated enzyme was performed on recombinant human GST-SGK2α fusion protein. Typically, the GST-SGK2α preparations were found to exhibit protein phosphotransferase activity in the order of about 200 pmol/min/ug in the presence of 50 uM of [γ-³²P]-ATP and 162 μM PKB substrate peptide (amino acid sequence: CKRPRAASFAE) during a 15 minute reaction at ambient temperature.

B. SGK2α In Vitro Kinase Assay

Compounds of formula (I) were tested in the following assay for their ability to inhibit the activity of SGK2a. The desired in vitro potency of a particular inhibitor is such that the compound is useful as a therapeutic agent, i.e. in the nanomolar or micromolar range.

(i) Assay Description

Test compounds were lyophilized and stored at −20° C. Stock solutions were made by weighing out the compounds and dissolving them in dimethyl sulfoxide (DMSO) to a standard concentration, usually 20 mM, and stored at −20° C. The compounds were diluted to a starting intermediate concentration of 250 μM in 1% DMSO, then serially diluted across a row of a 96 well plate using serial 2 fold dilution steps. Diluted 100% DMSO was used as a negative control.

5 μL of each compound dilution were robotically pipetted to Costar™ serocluster plates maintaining the same plate layout. All assay mixtures consisted of the following volumes:

5 μL diluted compound

10 μL target enzyme preparation

1 μL substrate

5 μL assay ATP

The assay mixtures were then incubated 15 minutes at ambient temperature.

From each assay mixture, 10 μL of assay mixture was spotted onto Millipore Multiscreen-PH™ opaque plates and washed twice for 10 minutes in 1% phosphoric acid. The plates were dried at 40° C. for 30 minutes, then the substrate phosphate complexes were quantitated by scintillation counting. These Millipore plates are in a 96-well format with immobilized P81 phosphocellulose membranes in the wells. Both the phosphorylated and non-phosphorylated form of the substrate bind to the membrane while ATP (unincorporated phosphate) is removed in the subsequent wash steps.

(ii) Calculation of IC₅₀

Inhibition of the targets by the test compounds is measured by scintillation counting of the incorporation of radioactive phosphate onto a specific substrate which is immobilized onto a filter paper at the end of the assay. To provide meaningful measurements of inhibition, the assays are performed both in the absence and presence of specific and known inhibitors, and the amount of incorporated radioactivity is compared to provide a baseline measurement.

The “baseline activity” is the amount of radioactivity incorporated in the absence of a target inhibitor. The amount of radioactivity incorporated in the presence of a target inhibitor is called the “sample activity”, and the % inhibition is expressed by the following formula:
% inhibition=100−(sample activity/baseline activity*100)

and is usually expressed in conjunction with the compound concentration. By using a range of target inhibitor concentrations, the IC₅₀ of an inhibitor is estimated (i.e. the concentration at which enzymatic activity is reduced by 50%). The IC₅₀ of various inhibitors against a particular target can be compared, where a lower IC₅₀ indicates a more potent inhibitor.

TABLE 1
Results of In vitro Assay

| Compound | IC₅₀ |
|---|---|
| 2-Benzyl-1-ethyl-6,7-dimethoxy-2H-isoquinolin-3-one | 0.97 |

### Example 2

### Cell Proliferation

This procedure (Jelinkova, R. B. et al., “Antiproliferative effect of a lectin- and anti-Thy-1.2 antibody-targeted HPMA copolymer-bound doxorubicin on primary and metastatic human colourectal carcinoma and on human colourectal carcinoma transfected with the mouse Thy-1.2 gene”, Bioconjug. Chem. (2000), Vol. 11, No. 5, pp. 664-73) is used to assess the effect compounds have on various cell lines with respect to proliferation. The rate of anchorage-independent growth of various tumour cells is quantified by measuring the amount of free isotopic thymidine that has been incorporated into the cells over a period of time. The effect of any compound to inhibit the proliferation of various tumour cells could be used as an indication of its ability to prevent disease progression in cancer.

Cultured tumour cells are harvested cells as per normal procedures: i.e. trypsinize, centrifuge and count cells. A volume of 90 μL is used to seed 5,000 cells/well in a 96 well plate. Cells are incubated for 24 hours at 37° C. under 5% CO₂. After incubation, cells should be 80-90% confluent.

³H-thymidine (Amersham) is diluted in cell culture media to a concentration of 100 μCi/mL. The test compound is diluted in the thymidine broth to 10× the final desired concentration.

Then 10 μL of diluted compound is added to the 90 μL of cells already present in the 96-well plates. Six replicate wells are done per treatment in columns 2 to 11. Plates were mixed by rocking.

A known cytotoxic compound such as staurosporine is used in relatively high concentrations as a positive control in column 1. Diluted DMSO is used as a negative control in column 12. The plate is incubated exactly 24 hours at 37° C.

After incubation, plates are observed under the microscope for obvious cell death, abnormal cell shape, crystal formation of the compound, etc. Then 25 μL volume of cold 50% TCA is added slowly to the 100 μL volume already in each well, and incubated for 1-2 hours at 4° C. The plates are then washed 5× in tap water and allowed to dry completely (usually overnight) at ambient temperature. Finally, 100 μL of scintillation fluid is added to each well and the plates are counted in a Wallac™ 1450 Microbeta™ counter according to user manual instructions.

The amount of inhibition is determined by the following formula:
% inhibition=100−[(AVG treatment−AVG positive control)/100(AVG negative control−AVG positive control)]

The results for in vitro inhibition of cell proliferation at 50 μM of 2-Benzyl-1-ethyl-6,7-dimethoxy-2H-isoquinolin-3-one were 27% for H460 cells, 42% for HUVEC cells, and 67% for PC3 cells.

### Example 3

### Cytotoxicity Assay

This procedure is used to assess the effects compounds have on various cell lines with respect to cell viability. Cell viability is quantified using calcein AM and measuring its conversion to a fluorescent product (calcein) with a fluorimeter.

The principle of this assay is based on the presence of ubiquitous intracellular esterase activity found in live cells. By enzymatic reaction of esterase, non-fluorescent cell-permeant calcein AM is converted to the intensely fluorescent calcein. The polyanionic dye calcein is retained within live cells, producing a green fluorescence in live cells. It is a faster, safer, and better-correlated indicator of cytotoxicity than alternative methods (e.g. ³H-Thymidine incorporation). It should be noted that calcein AM is susceptible to hydrolysis when exposed to moisture. Therefore, prepare aqueous working solutions containing calcein AM immediately prior to use, and use within about one day.

A kit available to do this assay is “LIVE/DEAD® Viability/Cytotoxicity Kit (L-3224)” by Molecular Probes.

Cells were collected from tissue culture flasks and trypsinized, centrifuged, resuspended and counted. Cells were seeded to obtain 80-90% confluence (for normal cells, 10,000 cells/well (8000 cells/well for HUVEC cells)). A cell concentration of 110,000 cells/mL (88,000 cells/well for HUVEC cells) is prepared as 90 μL volume is used per well.

Using an 8-channel multi-dispense pipettor, cells were seeded in the central rows of the plate (Nunclon™ 96 well flat-bottom plate), leaving the peripheral top and bottom rows with same volume of media only. The plates were incubated at 37° C., 5% CO₂ overnight for approximately 24 hours.

For test compounds, cell culture media (e.g., RPMI+10% FBS), 10× compound solution of final desired concentration from 20 mM stock compounds was prepared.

10 μl of this 10× compound solution is added to the 90 μL of cells already present in the 96 well plates and a known cytotoxic compound from previous testing is used as a positive control. The negative control is 100% DMSO diluted to the same factor as the compounds.

The plates are incubated at 37° C. for approximately 24 hours, and media is aspirated after plates are spun at 2400 rpm for 10 min at ambient temperature. 100 μL of 1×DPBS (without calcium chloride, without magnesium chloride (GibcoBRL, cat#14190-144)) is added to each well.

The calcein AM solution is prepared by added 50 μg of calcein AM crystal (m.w.=994.87 g/mol, Molecular Probes, Eugene, Oreg.) and nnhydrous DMSO (Sigma Aldrich) to make 1 mM stock and diluting stock to 2× the final desired concentration in 1×DPBS just before the assay. 100 μL of this 2× was added to the 100 μL of DPBS in the wells and the plates are incubated at ambient temperature for 30 minutes. Fluorescence data was read and recorded (Fluoroskan Ascent® FL fluorimeter (excitation˜485 nm, emission˜527 nm)).

The values for replicates (usually six) are averaged and % inhibition is calculated as follows:
% inhibition=100−[(AVG treatment−AVG positive control)/(AVG negative control−AVG positive control)*100]

The results for in vitro cytotoxicity at 50 μM of 2-Benzyl-1-ethyl-6,7-dimethoxy-2H-isoquinolin-3-one were: 0% proliferation for HS27 cells; 4% for HUVEC cells; and 0% for LL-86 cells.

### Example 4

### Xenograft Study

This experiment tests the efficacy of test compounds on H460 subcutaneous xenograft alone and in combination with doxorubicin.

Athymic nude female mice are used for this experiment. A group of 60 mice are inoculated with five million H460 cells in 100 μL Matrigel™ (VWR Canada) excipient. Tumours are measured three times a week with digital calipers and the tumour volumes calculated. When tumours have reached an average size of 100 mm³, about two weeks after tumour implantation. At that time any nongrowing ‘outliers’ are removed so that animals can be distributed into groupings that are equal and statistically the same tumour mass, i.e. divided into six groups with about 10 mice per group.

Treatments with test compounds continue for about 20 days, and will be oral (gavage), intravenous, subcutaneous, or intraperitoneal depending on the known solubility of the test compound. A dose of 25 mg/kg is typical for such testing, but the dose selected will reflect the potency of the compound and the route of administration. Up to 200 mg/kg may be selected.

Positive controls may alternately be cisplatin or cyclophosphamide.

The study breakdown in tabular form:

| | | | | | | Dose | | |
|---|---|---|---|---|---|---|---|---|
| Group | Treatment | Dose | Route | Schedule | 2^{nd} Treatment | mg/kg | Route | Schedule |
| A | PTE | — | — | — | None | — | — | |
| B | Compound | 25 mg/kg | I.P. | Daily for | None | — | — | |
| | | | | 20 days | | | | |
| C | Vehicle | — | I.P. | Daily for | Doxorubicin | 5 | IV | Every 4 |
| | | | | 20 days | | | | days |
| D | Vehicle | — | I.P. | Daily for | Doxorubicin | 7 | IV | Every 4 |
| | | | | 20 days | | | | days |
| E | Compound | 25 mg/kg | I.P. | Daily for | Doxorubicin | 5 | IV | Every 4 |
| | | | | 20 days | | | | days |
| F | Compound | 25 mg/kg | I.P. | Daily for | Doxorubicin | 7 | IV | Every 4 |
| | | | | 20 days | | | | days |

At study termination, the mice are anesthetized 3 hours after the last dose of test compound, and plasma and tissues are harvested and frozen. Tumours are divided into the desired number of aliquots and fast frozen for later analysis.

### Example 5

### Peritoneal Macrophage Stimulation and Analysis

A. Establishment of inflammation assay panel.

Macrophages are important elements of innate immunity to infection and are among the first cell type in the immune response to be exposed to and activated by infectious agents. IFN-γ and LPS are potent activators of macrophages, priming them for a variety of biological effects. IFN-γ, initially secreted by NK and T cells in response to infection, converts macrophages from a resting to an activated state (inflammatory macrophages), priming them for antimicrobial activity manifested by increased killing of intracellular pathogens, and antigen processing and presentation to lymphocytes. The action of IFN-γ is synergized with the LPS second messenger, enhancing the stimulation of macrophages through the activation of NF-κB, that results in the transcriptional up-regulation of a number of genes involved in the cell-mediated immune response, including inducible nitric oxide synthase (iNOS). Activated macrophages are qualitatively different from quiescent macrophages. These differences are typically observed by an increased proliferation index, up-regulated expression of MHC-II, and production of various bioactive molecules. The latter biological effects are mediated by nitric oxide (NO) release and increased production of pro-inflammatory cytokines (IL-6, TNF-γ, IL-1). Primary macrophages derived from Balb/c and RAW 264.7 cells (Balb/c background) were used to establish in vitro inflammatory models with fast and reliable readouts.

B. Materials and Methods

1. Reagents.

The iNOS inhibitor NG-Monomethyl-L-arginine (L-NMMA) and murine rIFN-γ were purchased from Calbiochem, (San Diego, Calif.). Protein-free, phenol/water-extracted LPS (from E. coli serotype 0111:B4 0127:B8), Zymosan A, dexamethasone and hydrocortisone, sulfanilamide and N-(1-naphthyl)-ethylenediamine, were purchased from Sigma (St. Louis, Mo.). Human recombinant vascular endothelial growth factor (VEGF) was purchased from R&D Systems (Minneapolis, Minn.). Rabbit polyclonal antibody against active (phosphorylated) extracellular signal-regulated kinase (ERK), as well as horse radish peroxidase (HRP)-conjugated donkey anti-rabbit IgG were obtained from Promega (Madison, Wis.). ELISA dual-set kit for detection of IL-6 was purchased from PharMingen (San Diego, Calif.). Anti-murine iNOS/NOS type II and cyclooxygenase 2 (COX-2) antibodies were obtained from Transduction Laboratories (Lexington, Ky.).

Female, 6-12 wk of age, BALB/c mice were purchased from Harlan Inc. (Indianapolis, Ind.) and housed under fluorescent light for 12 h per day. Mice are housed and maintained in compliance with the Canadian Council on Animal Care standards.

2. Isolation of Primary Mouse Macrophages.

Peritoneal exudate macrophages were isolated by peritoneal lavage with ice-cold sterile physiological saline 24 hours after intraperitoneal injection of BALB/c mice with 0.5 mL of sterile Zymosan A (1 mg/0.5 mL 0.9% saline). Cells were washed, resuspended in RPMI 1640 supplemented with 1 mM D-glucose, 1 mM sodium pyrovate, 100 units/mL penicillin, 100 μg/mL streptomycin, and 5% FBS.

3. Treatment of Primary Macrophages.

Primary macrophages (1.5×10⁵ cells/well) were grown in 96-well plates (nitrite assay), or 6-well plates (2×10⁶ cells/well) for measurement of iNOS and COX-2 expression. Following 3 hours incubation, at 37° C., 5% CO₂ (allowing macrophages to attach) cells were stimulated with LPS (5 μg/mL) and IFN-γ (100 U/mL) in the absence or presence of various concentrations of test compounds (all treatments were replicated six times). Cells were incubated for an additional 24 hours, and cell free culture supernatants from each well were collected for NO and cytokine determination. The remaining cells were stained with crystal violet or MTS to determine effect of the test compounds on cell survival.

4. NO Production.

Following stimulation, the production of NO was determined by assaying culture supernatants for NO₂⁻, a stable reaction product of NO with molecular oxygen. Briefly, 100 μL of culture supernatant was reacted with an equal volume of Griess reagent at ambient temperature for 10 minutes. The absorbance at 550 nm was determined. All measurements were performed six times. The concentration of NO₂⁻ was calculated by comparison with a standard curve prepared using NaNO₂.

5. Western Blot Analysis.

After incubation with the indicated stimuli in the presence of inhibitors, cells (duplicate samples, 2×10⁶ cell/6-wells plate) were washed in PBS and lysed on ice in 60 μL of lysis buffer. The protein content of each sample was determined using the Bradford protein assay kit (Bio-Rad, Richmond, Calif.). Absorbance was measured at 750 nm with a Beckman DU530 spectrophotometer (Palo Alto, Calif.). Proteins were mixed with 45×SDS sample buffer. Following separation of proteins by SDS-PAGE, using 8% bis-acrylamide in the separation gel, the proteins were transferred from the gels onto PVDF membranes using a MiniProtean™ III Cell (Bio-Rad), at 100 V for 1.5 hours. Equal amounts of protein (5 μg) were loaded onto SDS-PAGE gels and examined by Western blot analysis with anti-Actin, anti-iNOS, anti-COX-2 murine monoclonal antibodies, according to the manufacturer's specifications (Transduction Laboratories). Primary antibodies, in 5% blocking buffer (5% NFM/TTBS), were incubated with blots 2 hours at RT or overnight at 4° C., followed by incubation with peroxidase-conjugated secondary antibody. Chemiluminescence substrates were used to reveal positive bands. The bands were exposed on X-ray films. The films are used to analyze the impact of inhibitors on expression of iNOS and Cox-2 compared to various controls and “house-keeping” protein (actin) concentration to control the protein loading and detect any non-specific effects on protein production. The Multi-Analyst™/PC system from BioRad was used to quantitate the bands of the expressed protein on the film. This version of Multi-Analyst™ is used with the Bio-Rad Gel Doc 1000™ imaging system. White light is chosen as the selected light source, thus the signal strength is measured in OD (optic density) units. The OD of each band is being subtracted to arrive at a global background area of the gel.

C. In Vitro Angiogenesis.

HUVEC cells cultured for 24 hours in M199 with 0.5% FCS were plated at 6×105 cells/well in 12-well plates pre-coated with 300 μL of Matrigel™ (10.7 mg/mL; Becton Dickinson) in M199 with 0.5% FCS in the presence of VEGF (1 ng/mL), and in the absence or presence of positive control (Z)-3-[2,4-dimethyl-5-(2-oxo-1,2-dihydroindol-3-ylidenemethyl)-1H-pyrrol-3-yl] propionic acid or various inhibitors. After 5 hours of incubation in a 5% CO₂-humidified atmosphere at 37° C., the three-dimensional organization of the cells was examined using an inverted photomicroscope. The cells were fixed with crystal violet (0.05% in 20% ethanol) and digitally photographed.

C. Enzyme Immunoassays for Mouse IL-6.

IL-6 levels were determined with PharMingen's OptEIA™ ELISA set developed using an anti-mouse IL-6 antibody pair and mouse rIL-6 standard (PharMingen). MaxiSorp™ F16 multiwell strips (Nunc, Roskilde, Denmark) were coated with anti-mouse IL-6 capture antibody (at recommended concentration) in 0.1 M NaHCO₃, pH 9.5, 100 μL/well, overnight at 4° C. Plates were washed three times with 0.05% Tween 20 in PBS (PBST) and blocked for 1 hour at ambient temperature with 200 μL/well of 10% FCS in PBS (blocking and dilution buffer). Plates were washed three times with PBST and duplicate samples (100 μL/well) or standards (100 μL/well) in diluent buffer were incubated for 2 hours at ambient temperature. Plates were washed five times with PBST and incubated with biotinylated anti-mouse IL-6 and avidin-HRP conjugate (at concentrations recommended by the manufacturer) for 1 hour at ambient temperature. Plates were washed seven times with PBST and 100 μL of 3,3′5,5′ tetramethylbenzidine substrate solution (TMB substrate reagent set, BD PharMingen) was added to each well. After 15-30 minute incubation at ambient temperature, colour development was terminated by adding 50 μL of 2 N H₂SO₄ (Sigma). Absorbance was read at 450 nm with an EL 312e™ microplate reader (or equivalent). The lower limit of detection for IL-6 was 15.6 pg/mL.

All of the U.S. patents, U.S. patent application publications, U.S. patent applications, foreign patents, foreign patent applications and non-patent publications referred to in this specification and/or listed in the Application Data Sheet are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A pharmaceutical composition useful in treating cancer, inflammation or a hyperproliferative disorder in a human, wherein the pharmaceutical composition comprises a pharmaceutically acceptable carrier, diluent or excipient and a compound of formula (I): 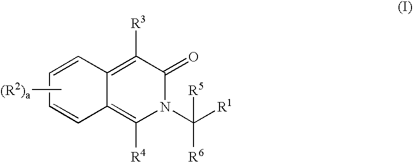
wherein:
a is 0 to 4;
R¹ is carbocyclyl or heterocyclyl;
each R² is selected from the group consisting of hydrogen, alkyl, alkenyl, aryl, aralkyl, aralkenyl, halo, haloalkyl, haloalkenyl, nitro, cyano, cycloalkyl, cycloalkylalkyl, cycloalkylalkenyl, heterocyclyl, heterocyclylalkyl, —OR⁷, —C(O)OR⁷, —C(O)N(R⁷)₂, —N(R⁷)₂, —N(R⁷)C(O)OR⁸, —N(R⁷)C(O)R⁷, —N(R⁷)C(O)N(R⁷)₂, —R⁹—N═N—O—R⁸, —S(O)ₚR⁷ (where p is 0 to 2), and —S(O)ₚN(R⁷)₂ (where p is 0 to 2);
R³ and R⁴ are each independently selected from the group consisting of hydrogen, alkyl, alkenyl, aryl, aralkyl, aralkenyl, halo, haloalkyl, haloalkenyl, nitro, cyano, cycloalkyl, cycloalkylalkyl, cycloalkylalkenyl, heterocyclyl, heterocyclylalkyl, —OR⁷, —C(O)OR⁷, —C(O)N(R⁷)₂, —N(R⁷)₂, —N(R⁷)C(O)OR⁸, —N(R⁷)C(O)N(R⁷)₂, —N(R⁷)C(O)R⁷, —R⁹—N═N—O—R⁸, —S(O)ₚR⁷ (where p is 0 to 2), and —S(O)ₚN(R⁷)₂ (where p is 0 to 2);
R⁵ and R⁶ are each independently selected from the group consisting of hydrogen, alkyl, alkenyl, haloalkyl, haloalkenyl, cycloalkyl, cycloalkenyl and heterocyclylalkyl;
each R⁷ is independently selected from the group consisting of hydrogen, alkyl, alkenyl, haloalkyl, haloalkenyl, aryl, aralkyl, aralkenyl, cycloalkyl, cycloalkylalkyl and cycloalkylalkenyl;
each R⁸ is independently selected from the group consisting of hydrogen, alkyl, alkenyl, haloalkyl, haloalkenyl, aralkyl, aralkenyl, cycloalkyl, cycloalkylalkyl and cycloalkylalkenyl; and
R⁹ is a bond or a straight or branched alkylene or alkenylene chain;
as a single stereoisomer, a mixture of stereoisomers, or as a racemic mixture of stereoisomers; or as a solvate or polymorph; or as a pharmaceutically acceptable salt thereof,
with the proviso that R¹ can not be unsubstituted phenyl when all of the following occur:
(i) a is 2 and one R² is methoxy in the 6-position of the isoquinolone ring and the other R² is methoxy in the 7-position of the isoquinolone ring; and
(ii) R³, R⁵ and R⁶ are all hydrogen, and
(iii) R⁴ is 3,4-dimethoxybenzyl.

2.-39. (canceled)

40. A method of treating cancer, inflammation or a hyperproliferative disorder in a mammal, which method comprises administering to the mammal in need thereof a therapeutically effective amount of a compound of formula (I): 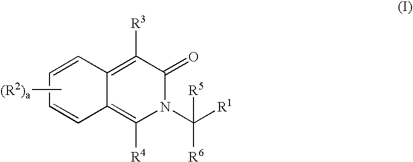
wherein:
a is 0 to 4;
R¹ is carbocyclyl or heterocyclyl;
each R² is selected from the group consisting of hydrogen, alkyl, alkenyl, aryl, aralkyl, aralkenyl, halo, haloalkyl, haloalkenyl, nitro, cyano, cycloalkyl, cycloalkylalkyl, cycloalkylalkenyl, heterocyclyl, heterocyclylalkyl, —OR⁷, —C(O)OR⁷, —C(O)N(R⁷)₂, —N(R⁷)₂, —N(R⁷)C(O)N(R⁷)₂, —N(R⁷)C(O)OR⁸, —N(R⁷)C(O)R⁷, —R⁹—N═N—O—R⁸, —S(O)ₚR⁷ (where p is 0 to 2), and —S(O)ₚN(R⁷)₂ (where p is 0 to 2);
R³ and R⁴ are each independently selected from the group consisting of hydrogen, alkyl, alkenyl, aryl, aralkyl, aralkenyl, halo, haloalkyl, haloalkenyl, nitro, cyano, cycloalkyl, cycloalkylalkyl, cycloalkylalkenyl, heterocyclyl, heterocyclylalkyl, —OR⁷, —C(O)OR⁷, —C(O)N(R⁷)₂, —N(R⁷)₂, —N(R⁷)C(O)N(R⁷)₂, —N(R⁷)C(O)OR⁸, —N(R⁷)C(O)R⁷, —R⁹—N═N—O—R⁸, —S(O)ₚR⁷ (where p is 0 to 2), and —S(O)ₚN(R⁷)₂ (where p is 0 to 2);
R⁵ and R⁶ are each independently selected from the group consisting of hydrogen, alkyl, alkenyl, haloalkyl, haloalkenyl, cycloalkyl, cycloalkenyl and heterocyclylalkyl; each R⁷ is independently selected from the group consisting of hydrogen, alkyl, alkenyl, haloalkyl, haloalkenyl, aryl, aralkyl, aralkenyl, cycloalkyl, cycloalkylalkyl and cycloalkylalkenyl;
each R⁸ is independently selected from the group consisting of hydrogen, alkyl, alkenyl, haloalkyl, haloalkenyl, aralkyl, aralkenyl, cycloalkyl, cycloalkylalkyl and cycloalkylalkenyl; and
R⁹ is a bond or a straight or branched alkylene or alkenylene chain;
as a single stereoisomer, a mixture of stereoisomers, or as a racemic mixture of stereoisomers; or as a solvate or polymorph; or as a pharmaceutically acceptable salt thereof.

41. (canceled)

42. The method according to claim 40 wherein the cancer or inflammation is associated with hyperproliferation or cell survival.

43. The method according to claim 40 wherein the hyperproliferative disease, cancer or inflammation is associated with the activity of SGK.

44. (canceled)

45. A method of treating a mammal having a disorder or condition associated with hyperproliferation and cell survival, wherein said method comprises administering to the mammal having the disorder or condition a therapeutically effective amount of a compound of formula (I): 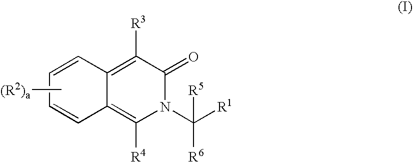
wherein:
a is 0 to 4;
R¹ is carbocyclyl or heterocyclyl;
each R² is selected from the group consisting of hydrogen, alkyl, alkenyl, aryl, aralkyl, aralkenyl, halo, haloalkyl, haloalkenyl, nitro, cyano, cycloalkyl, cycloalkylalkyl, cycloalkylalkenyl, heterocyclyl, heterocyclylalkyl, —OR⁷, —C(O)OR⁷, —C(O)N(R⁷)₂, —N(R⁷)₂, —N(R⁷)C(O)N(R⁷)₂, —N(R⁷)C(O)OR⁸, —N(R⁷)C(O)R⁷, —R⁹—N═N—O—R⁸, —S(O)ₚR⁷ (where p is 0 to 2), and —S(O)ₚN(R⁷)₂ (where p is 0 to 2);
R³ and R⁴ are each independently selected from the group consisting of hydrogen, alkyl, alkenyl, aryl, aralkyl, aralkenyl, halo, haloalkyl, haloalkenyl, nitro, cyano, cycloalkyl, cycloalkylalkyl, cycloalkylalkenyl, heterocyclyl, heterocyclylalkyl, —OR⁷, —C(O)OR⁷, —C(O)N(R⁷)₂, —N(R⁷)₂, —N(R⁷)C(O)N(R⁷)₂, —N(R⁷)C(O)OR⁸, —N(R⁷)C(O)R⁷, —R⁹—N═N—O—R⁸, —S(O)ₚR⁷ (where p is 0 to 2), and —S(O)ₚN(R⁷)₂ (where p is 0 to 2);
R⁵ and R⁶ are each independently selected from the group consisting of hydrogen, alkyl, alkenyl, haloalkyl, haloalkenyl, cycloalkyl, cycloalkenyl and heterocyclylalkyl;
each R⁷ is independently selected from the group consisting of hydrogen, alkyl, alkenyl, haloalkyl, haloalkenyl, aryl, aralkyl, aralkenyl, cycloalkyl, cycloalkylalkyl and cycloalkylalkenyl;
each R⁸ is independently selected from the group consisting of hydrogen, alkyl, alkenyl, haloalkyl, haloalkenyl, aralkyl, aralkenyl, cycloalkyl, cycloalkylalkyl and cycloalkylalkenyl; and
R⁹ is a bond or a straight or branched alkylene or alkenylene chain;
as a single stereoisomer, a mixture of stereoisomers, or as a racemic mixture of stereoisomers; or as a solvate or polymorph; or as a pharmaceutically acceptable salt thereof.

46. The method according to claim 40 or claim 45 wherein the mammal is a human.

47. A method of treating a mammalian cell with a compound of formula (I): 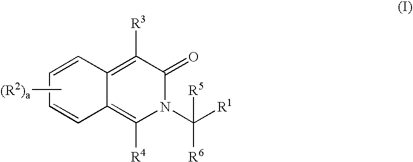
wherein:
a is 0 to 4;
R¹ is carbocyclyl or heterocyclyl;
each R² is selected from the group consisting of hydrogen, alkyl, alkenyl, aryl, aralkyl, aralkenyl, halo, haloalkyl, haloalkenyl, nitro, cyano, cycloalkyl, cycloalkylalkyl, cycloalkylalkenyl, heterocyclyl, heterocyclylalkyl, —OR⁷, —C(O)OR⁷, —C(O)N(R⁷)₂, —N(R⁷)₂, —N(R⁷)C(O)N(R⁷)₂, —N(R⁷)C(O)OR⁸, —N(R⁷)C(O)R⁷, —R⁹—N═N—O—R⁸, —S(O)ₚR⁷ (where p is 0 to 2), and —S(O)ₚN(R⁷)₂ (where p is 0 to 2);
R³ and R⁴ are each independently selected from the group consisting of hydrogen, alkyl, alkenyl, aryl, aralkyl, aralkenyl, halo, haloalkyl, haloalkenyl, nitro, cyano, cycloalkyl, cycloalkylalkyl, cycloalkylalkenyl, heterocyclyl, heterocyclylalkyl, —OR⁷, —C(O)OR⁷, —C(O)N(R⁷)₂, —N(R⁷)₂, —N(R⁷)C(O)N(R⁷)₂, —N(R⁷)C(O)OR⁸, —N(R⁷)C(O)R⁷, —R⁹—N═N—O—R⁸, —S(O)ₚR⁷ (where p is 0 to 2), and —S(O)ₚN(R⁷)₂ (where p is 0 to 2);
R⁵ and R⁶ are each independently selected from the group consisting of hydrogen, alkyl, alkenyl, haloalkyl, haloalkenyl, cycloalkyl, cycloalkenyl and heterocyclylalkyl;
each R⁷ is independently selected from the group consisting of hydrogen, alkyl, alkenyl, haloalkyl, haloalkenyl, aryl, aralkyl, aralkenyl, cycloalkyl, cycloalkylalkyl and cycloalkylalkenyl;
each R⁸ is independently selected from the group consisting of hydrogen, alkyl, alkenyl, haloalkyl, haloalkenyl, aralkyl, aralkenyl, cycloalkyl, cycloalkylalkyl and cycloalkylalkenyl; and
R⁹ is a bond or a straight or branched alkylene or alkenylene chain;
as a single stereoisomer, a mixture of stereoisomers, or as a racemic mixture of stereoisomers; or as a solvate or polymorph; or as a pharmaceutically acceptable salt thereof, wherein the method comprises administering the compound of formula (I) to a mammalian cell and the compound of formula (I) is capable of inhibiting the activity of SGK within the mammalian cell.

48. The method of claim 47 wherein the mammalian cell is treated in vitro.

49. The method of claim 47 wherein the mammalian cell is treated in vivo.

50. The method of claim 47 wherein the inhibition of activity results in a reduction of cell survival.

51. The method of claim 47 wherein the inhibition of activity results in a reduction of cell division.

52. The method of claim 47, wherein the inhibition of activity results in apoptosis.

53. The method of claim 47, wherein the inhibition of activity results in control of tumour growth.

54. The method or pharmaceutical composition of claim 1 or claim 40 wherein R¹ is carbocyclyl.

55. The method or pharmaceutical composition of claim 1 or claim 40 wherein R¹ is aryl.

56. The method or pharmaceutical composition of claim 1 or claim 40 wherein R¹ is cycloalkyl.

57. The method or pharmaceutical composition of claim 1 or claim 40 wherein R¹ is heterocyclyl.

58. The method or pharmaceutical composition of claim 1 or claim 40 wherein at least one R² is hydrogen, alkyl, alkenyl, cycloalkyl, cycloalkylalkyl or cycloalkylalkenyl.

59. The method or pharmaceutical composition of claim 1 or claim 40 wherein at least one R² is aryl, aralkyl or aralkenyl.

60. The method or pharmaceutical composition of claim 1 or claim 40 wherein at least one R² is halo, haloalkyl or haloalkenyl.

61. The method or pharmaceutical composition of claim 1 or claim 40 wherein at least one R² is nitro, cyano, —N(R⁷)₂, —N(R⁷)C(O)OR⁸, —N(R⁷)C(O)R⁷ or —R⁹—N═N—O—R⁸.

62. The method or pharmaceutical composition of claim 1 or claim 40 wherein at least one R² is heterocyclyl or heterocyclylalkyl.

63. The method or pharmaceutical composition of claim 1 or claim 40 wherein at least one R² is —C(O)OR⁷ or —C(O)N(R⁷)₂.

64. The method or pharmaceutical composition of claim 1 or claim 40 wherein at least one R² is —OR⁷, —S(O)ₚR⁷ (where p is 0 to 2), or —S(O)ₚN(R⁷)₂ (where p is 0 to 2).

65. The method or pharmaceutical composition of claim 1 or claim 40 wherein R³ is hydrogen, alkyl, alkenyl, halo, haloalkyl, haloalkenyl, cycloalkyl, cycloalkylalkyl or cycloalkylalkenyl.

66. The method or pharmaceutical composition of claim 1 or claim 40 wherein R³ is aryl, aralkyl or aralkenyl.

67. The method or pharmaceutical composition of claim 1 or claim 40 wherein R³ is nitro, cyano, —N(R⁷)₂, —N(R⁷)C(O)OR⁸, —N(R⁷)C(O)R⁷ or —R⁹—N═N—O—R⁸.

68. The method or pharmaceutical composition of claim 1 or claim 40 wherein R³ is heterocyclyl or heterocyclylalkyl.

69. The method or pharmaceutical composition of claim 1 or claim 40 wherein R³ is —C(O)OR⁷ or —C(O)N(R⁷)₂.

70. The method or pharmaceutical composition of claim 1 or claim 40 wherein R³ is —OR⁷, —S(O)ₚR⁷ (where p is 0 to 2) or —S(O)ₚN(R⁷)₂ (where p is 0 to 2).

71. The method or pharmaceutical composition of claim 1 or claim 40 wherein R⁴ is hydrogen, alkyl, alkenyl, halo, haloalkyl, haloalkenyl, cycloalkyl, cycloalkylalkyl or cycloalkylalkenyl.

72. The method or pharmaceutical composition of claim 1 or claim 40 wherein R⁴ is aryl, aralkyl or aralkenyl.

73. The method or pharmaceutical composition of claim 1 or claim 40 wherein R⁴ is nitro, cyano, —N(R⁷)₂, —N(R⁷)C(O)OR⁸, —N(R⁷)C(O)R⁷ or —R⁹—N═N—O—R⁸.

74. The method or pharmaceutical composition of claim 1 or claim 40 wherein R⁴ is heterocyclyl or heterocyclylalkyl.

75. The method or pharmaceutical composition of claim 1 or claim 40 wherein R⁴ is —C(O)OR⁷ or —C(O)N(R⁷)₂.

76. The method or pharmaceutical composition of claim 1 or claim 40 wherein R⁴ is —OR⁷, —S(O)ₚR⁷ (where p is 0 to 2) or —S(O)ₚN(R⁷)₂ (where p is 0 to 2).

77. The method or pharmaceutical composition of claim 1 or claim 40 wherein R⁵ and R⁶ are each independently selected from the group consisting of hydrogen, alkyl or haloalkyl.
